(19) United States
(12) Patent Application Publication
(10) Pub. No.: US 2024/0424304 A1
(43) Pub. Date: Dec. 26, 2024

(54) SYSTEMS, DEVICES, AND METHODS FOR HIS BUNDLE CARDIAC PACING

(71) Applicant: EPQuant, LLC, Haverford, PA (US)

(72) Inventor: Holly Marie Sinnott, Springfield, PA (US)

(21) Appl. No.: 18/644,501

(22) Filed: Apr. 24, 2024

Related U.S. Application Data
(63) Continuation of application No. 18/188,276, filed on Mar. 22, 2023, now abandoned.
Application No. 18/188,276 is a continuation of application No. 16/558,854, filed on Sep. 3, 2019, now abandoned.
(60) Provisional application No. 62/726,857, filed on Sep. 4, 2018.
(60) Provisional application No. 62/726,277, filed on Sep. 2, 2018.

(51) Int. Cl. A61N 1/362; A61N 1/375

(52) U.S. Cl. CPC A61N 1/362; A61N 1/37512; A61N 1/37518; A61N 1/3756

(57) ABSTRACT

The present disclosure relates to devices and methods for cardiac pacing therapy. Disclosed herein are methods for His bundle cardiac pacing; cardiac leads and leadless cardiac pacemakers that enables pacing and sensing of the His bundle as well as the right atrium and right ventricle; and delivery sheaths for placing the cardiac lead or leadless cardiac pacemaker in the heart. The devices and methods disclosed increase the success at which His bundle pacing can be implemented.

### CROSS REFERENCE TO RELATED APPLICATIONS

This patent application is a continuation of U.S. patent application Ser. No. 18/188,276, filed on Mar. 22, 2023, which is a continuation of U.S. patent application Ser. No. 16/558,854, filed on Sep. 3, 2019, which claims priority to U.S. Provisional Patent Application No. 62/726,277, filed on Sep. 2, 2018 and U.S. Provisional Patent Application No. 62/726,857, filed on Sep. 4, 2018; the entire contents of each of the aforementioned applications is incorporated herein by reference.

### FIELD OF THE INVENTION

The present disclosure relates generally to cardiac pacing, and specifically to systems, devices, including cardiac pacing leads and leadless pacemakers, and methods for His bundle pacing.

### BACKGROUND OF THE INVENTION

Cardiac pacemakers are a commonly used treatment method to sustain an appropriate heart rate in patients who suffer from cardiac conduction system disorders and arrhythmias. The standard treatment method for patients requiring cardiac pacing involves implantation of a pulse generator, which is connected to pacemaker lead(s) implanted into the right atrium, right ventricle, or both the right atrium and right ventricle. The pacemaker system is then able to sense and pace the chamber(s) of the heart. The standard pacing method does not activate the native conduction system, and therefore, results in less efficient electrical and mechanical activation of the heart. Standard right ventricular cardiac pacing may result in pacing-induced cardiomyopathy and heart failure (Sharma, Ellenbogen, & Trohman, 2017).

His bundle pacing is a method which activates the native conduction system and promotes a more efficient heart contraction than standard right ventricular pacing. To perform His bundle pacing with the current available tools, the physician deploys a pacemaker lead with or without a sheath, into the region of the His bundle. These leads are generally implanted with the distal tip of the lead directed at the His bundle. The distal tip of the lead has a helix fixation mechanism that is deployed approximately perpendicular to the endocardial surface into the endocardium. When the lead is properly implanted for His bundle pacing, the helix is both a fixation mechanism and a pacing electrode.

His bundle pacing is technically challenging to implement due to the limitations of the current tools and methods. Leads currently available have a single distal electrode which must be directly deployed into a small target region to achieve His bundle pacing. The target region is approximately one square centimeter. The challenge of engaging the small target region with the currently available leads makes His bundle pacing procedures both difficult and time-consuming.

Recent and more advanced approaches to heart pacemakers include leadless pacemakers which provide distinct advantages to standard pacemakers. Unlike standard pacemakers, leadless pacemakers are implanted directly into the right ventricle and do not require a surgical pocket to be made for placement of the pacemaker. Leadless pacemakers contain a pulse generator with a built-in battery and electrodes. The physician deploys the pacemaker via a catheter into the right ventricle. Some advantages of leadless pacemakers include avoidance of pacemaker erosion, pain at the pacemaker site, undesirable cosmetics, lead placement complications; risk of infection has been shown to be lower with leadless pacemakers.

Improved systems, devices, and methods are necessary to increase the success of His bundle pacing procedures. The disclosed systems, devices, and methods overcome the current challenges of His bundle pacing by increasing the procedural success and case at which this therapy can be administered by physicians.

### BRIEF SUMMARY OF THE INVENTION

The appended claims define this application. The present disclosure summarizes aspects of the embodiments and should not be used to limit the claims. Other implementations are contemplated in accordance with the techniques described herein, as will be apparent to one having ordinary skill in the art upon examination of the following drawings and detailed description, and these implementations are intended to be within the scope of this application.

In one aspect, the present disclosure provides methods for His bundle pacing. In certain embodiments, method comprises selectively delivering an electrical stimulation pulse to one or more individual electrodes of an approximately linear array of electrodes, wherein the approximately linear array of electrodes is fixated to intersect the His bundle. In some such embodiments, the approximately linear array of electrodes is fixated to cardiac tissue by a tine, an extendible helix, or a combination thereof. In some such embodiments, the approximately linear array of electrodes comprises at least four, preferably at least eight, electrodes. In some such embodiments, the approximately linear array of electrodes is fixated approximately orthogonal to the His bundle. In some such embodiments, the approximately linear array of electrodes is disposed on a distal section of a pacing lead. In some such embodiments, the approximately linear array of electrodes is disposed on a leadless pacemaker.

In another aspect, the present disclosure provides a pacing lead comprising a distal section having a distal fixation mechanism, a distal section having a plurality of electrodes along the side of the lead, and a proximal section having an adapted end with multiple electrodes. The distal section may be implanted in the region of the His bundle and the proximal adapted end is used to connect to a pulse generator.

In another aspect, the present disclosure provides a pacing lead comprising a distal section having a plurality of electrodes and a fixation mechanism on a side of the lead body, and a proximal section having an end adapted to be connected to a pulse generator. In certain embodiments, the plurality of electrodes are arranged approximately linearly along the distal section of the lead. In certain embodiments, the fixation mechanism comprises an active tine or an extendible helix or a combination thereof. In certain embodiments, the plurality of electrodes comprises at least four, preferably at least eight, electrodes. In certain embodiments, at least one electrode of the plurality of electrodes is a dome electrode, a penetrating electrode, or an active tine electrode.

In another aspect, the present disclosure provides a lead with multiple electrodes, preferably arranged in an approximately linear array, along the side of the lead. For example, the lead may include eight pacing electrodes; in an alternative embodiment, the lead includes four pacing electrodes. In certain embodiments, the approximately linear electrode array is on the distal section of the lead. In some such embodiments, the approximately linear electrode array is along the side of the distal section of the lead.

Lead designs having an approximately linear arrangement of electrodes are referred to throughout this disclosure a “linear pacing lead.” In operation, the function of the linear pacing lead is to obtain sensing and pacing in the region of the His bundle. When optimally placed, certain linear pacing leads are capable of sensing and pacing the atrium, His bundle, and the right ventricle. Thus, a linear pacing lead may also be called an Atrial-His-Ventricular lead or AHV lead. In certain embodiments, such an AHV lead is implanted in the heart and fixation mechanisms maintain contact between the multiple electrodes and the endocardium. In certain embodiments, when implanted, the distal section of the AHV lead is fixated to three anatomical structures, allowing electrodes to interact with those structures. In some such embodiments, the distal section of the AHV lead includes two or more fixation mechanisms to engage the base of the right ventricle, the region of the His bundle, and/or the right atrium. The distal aspect of the distal section is associated with the right ventricle; the mid aspect of the distal section is associated with the His bundle; the proximal aspect of the distal section is associated with the right atrium.

In another aspect, the present disclosure provides a delivery sheath for delivery of the linear pacing lead, and particularly the AHV lead, to the described anatomy. The delivery sheath guides the lead and facilitates deployment of the fixation mechanisms to the endocardium for lead stabilization.

In yet another aspect, the present disclosure provides a pacemaker comprising a fixation mechanism and a plurality of electrodes along the side of the pacemaker. The pacemaker may be implanted in the region of the His bundle.

In another aspect, the present disclosure provides a leadless pacemaker comprising a plurality of electrodes along the side of the body of the pacemaker and a fixation mechanism. In certain embodiments, the plurality of electrodes are arranged approximately linearly along the longitudinal axis of the pacemaker body. In certain embodiments, the fixation mechanism comprises an active tine or an extendible helix or a combination thereof. In certain embodiments, the plurality of electrodes comprises at least four, preferably at least eight, electrodes. In certain embodiments, at least one electrode of the plurality of electrodes is a dome electrode, a penetrating electrode, or an active tine electrode.

In still another aspect, the present disclosure provides a leadless pacemaker with multiple electrodes arranged in an approximately linear array on the side of the pacemaker. For example, the leadless pacemaker may include eight pacing electrodes; in an alternative embodiment, the leadless pacemaker includes four pacing electrodes.

Pacemaker designs having an approximately linear arrangement of electrodes are referred to throughout this disclosure a “linear leadless pacemaker.” In operation, the function of the linear leadless pacemaker is to obtain sensing and pacing in the region of the His bundle. When optimally placed, certain linear leadless pacemakers are capable of sensing and pacing the atrium, His bundle, and the right ventricle. When some embodiments of the linear leadless pacemaker are implanted in the heart, fixation mechanisms maintain contact between the multiple electrodes and the endocardium.

In certain embodiments, when implanted, the linear leadless pacemaker is fixated to three anatomical structures, allowing electrodes to interact with those structures. In some such embodiments, the linear leadless pacemaker includes two or more fixation mechanisms to engage the base of the right ventricle, the region of the His bundle, and/or the right atrium. The distal section of the linear leadless pacemaker is associated with the right ventricle; the mid section of the linear leadless pacemaker is associated with the His bundle; the proximal section of the linear leadless pacemaker is associated with the right atrium.

In another aspect, the present disclosure provides a delivery sheath for delivery of the linear leadless pacemaker to the described anatomy. The delivery sheath guides the linear leadless pacemaker and facilitates deployment of the fixation mechanisms to the endocardium for pacemaker stabilization.

### BRIEF DESCRIPTION OF THE DRAWINGS

The summary, as well as the following detailed description, is further understood when read in conjunction with the appended drawings. For the purpose of illustrating the disclosed methods and devices, exemplary embodiments of the methods and devices are shown in the drawings, however, the methods and devices are not limited to the specific embodiments disclosed. In the drawings:

FIG. 1A illustrates a frontal view of the cardiac conduction system of the heart.

FIG. 1B illustrates a frontal view of the His bundle region.

FIG. 2A illustrates a frontal view of an exemplary AHV lead positioned in the heart and connected to a pulse generator.

FIG. 2B illustrates a frontal view of an exemplary linear leadless pacemaker in the heart in the region of the His bundle.

FIG. 2C illustrates the electrograms related to the anatomy in FIG. 2A or FIG. 2B.

FIG. 3A illustrates a frontal view of an exemplary full AHV lead, with the distal section of the AHV lead having a linear array of eight electrodes with active tine fixation.

FIG. 3B illustrates a frontal view of the distal section of the exemplary AHV lead having a linear array of eight electrodes with active tine fixation.

FIG. 3C illustrates a side view of the distal section of the exemplary AHV lead having a linear array of eight electrodes with active tine fixation.

FIG. 4A illustrates a frontal view of an exemplary full AHV lead, with the distal section of the AHV lead having a linear array of eight electrodes with active tine and active helix fixation.

FIG. 4B illustrates a frontal view of the distal section of the exemplary AHV lead having a linear array of eight electrodes with active tine and active helix fixation.

FIG. 4C illustrates a side view of the distal section of the exemplary AHV lead having a linear array of eight electrodes with active tine and active helix fixation

FIG. 5A illustrates a frontal view of an exemplary full AHV lead, with the distal section of an AHV lead having a linear array of eight electrodes with active helix fixation.

FIG. 5B illustrates a frontal view of the distal section of the exemplary AHV lead having a linear array of eight electrodes with active helix fixation.

FIG. 5C illustrates a side view of the distal section of the exemplary AHV lead having a linear array of eight electrodes with active helix fixation.

FIG. 6A illustrates a frontal view of an exemplary linear pacing lead, with the distal section of the linear pacing lead having a linear array of four electrodes with active tine fixation.

FIG. 6B illustrates a frontal view of the distal section of the exemplary linear pacing lead having a linear array of four electrodes with active tine fixation.

FIG. 6C illustrates a side view of the distal section of the exemplary linear pacing lead having a linear array of four electrodes with active tine fixation.

FIG. 7 illustrates a side view of an exemplary delivery sheath for an exemplary linear pacing lead.

FIG. 8A illustrates a frontal view of an exemplary linear leadless pacemaker having a linear array of eight electrodes with active tine fixation.

FIG. 8B illustrates a side view of the exemplary linear leadless pacemaker having a linear array of eight electrodes with active tine fixation.

FIG. 8C illustrates a proximal end view of the exemplary linear leadless pacemaker.

FIG. 9A illustrates a frontal view of an exemplary linear leadless pacemaker having a linear array of eight electrodes with active tine and active helix fixation.

FIG. 9B illustrates a side view of the exemplary linear leadless pacemaker having a linear array of eight electrodes with active tine and active helix fixation.

FIG. 9C illustrates a proximal end view of the exemplary linear leadless pacemaker.

FIG. 10A illustrates a frontal view of an exemplary linear leadless pacemaker having a linear array of eight electrodes with active helix fixation.

FIG. 10B illustrates a side view of the exemplary linear leadless pacemaker having a linear array of eight electrodes with active helix fixation.

FIG. 10C illustrates a proximal end view of the exemplary linear leadless pacemaker.

FIG. 11 illustrates a side view of an exemplary delivery sheath for an exemplary linear leadless pacemaker.

FIG. 12A illustrates a side translucent view of an exemplary linear leadless pacemaker with active tine fixation prior to retraction of the slider during deployment.

FIG. 12B illustrates a side translucent view of the exemplary linear leadless pacemaker with active tine fixation as the slider is retracted during deployment.

FIG. 13A illustrates a frontal view of the distal aspect of an exemplary smart lead.

FIG. 13B illustrates a side view of the distal aspect of the exemplary smart lead.

### DETAILED DESCRIPTION OF THE INVENTION

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated. Thus, the following examples are illustrative only and are not a limitation on the present invention.

The disclosed methods and devices may be understood more readily by reference to the following detailed description taken in connection with the accompanying figures, which form a part of this disclosure. It is to be understood that the disclosed methods and devices are not limited to the specific methods and devices described and/or shown herein, and that the terminology used herein is for the purpose of describing particular embodiments by way of example only and is not intended to be limiting of the claimed methods and devices.

Unless specifically stated otherwise, any description as to a possible mechanism or mode of action or reason for improvement is meant to be illustrative only, and the disclosed methods and devices are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of +10% or less, variations of +5% or less, variations of +1% or less, variations of +0.5% or less, or variations of +0.1% or less from the specified value.

It is to be appreciated that certain features of the disclosed methods and devices which are, for clarity, described herein in the context of separate embodiments, may also be provided in combination in a single embodiment. Conversely, various features of the disclosed methods and devices that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

### I. Methods

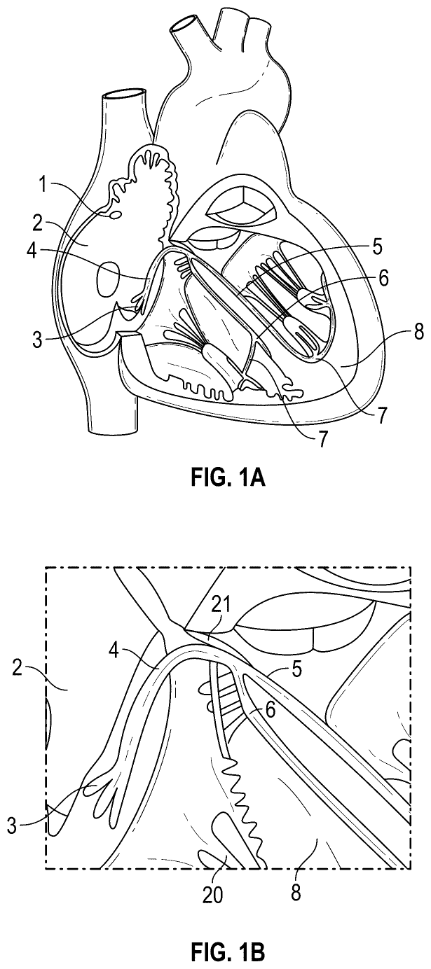

FIG. 1A relates to cardiac anatomy relevant to His bundle pacing. Depicted are the sinus node 1, the right atrium tissue 2, the AV node 3, the His bundle 4, the left bundle branch 5, the right bundle branch 6, the Purkinje system 7, and the right ventricular tissue 8. In sinus rhythm with normal AV conduction, there is sequential activation which originates from the sinus node. The signal then travels to the right atrial tissue, followed by the AV node, the His bundle, the left and right bundle branches, the Purkinje system, and ultimately the ventricular tissue. States of disease may affect any of the components of this system and ultimately require cardiac pacing to sustain an appropriate heart rate. It has been found that pacing the His bundle results in a more synchronous and efficient ventricular contraction than both right ventricular pacing and biventricular pacing. Thus, it is very desirable to have an efficient method for pacing the His bundle to result in ventricular activation.

FIG. 1B is a more detailed review of the anatomy in the region of the His bundle. In FIG. 1B, the right atrium tissue 2, the AV node 3, the His bundle 4, the left bundle branch 5, the right bundle branch 6, and the right ventricular tissue 8 are components which are also included in FIG. 1A. Additional components of this region include the tricuspid valve 20 and membranous septum 21. His bundle pacing may be achieved within the His bundle at any location with proximity to the His bundle. In most cases, the His bundle courses along the lower border of the membranous septum. At the distal end of the membranous septum, the His bundle splits into the right and left bundles with a left bundle penetrating the membranous septum. His bundle pacing is intended to capture the His bundle before conduction fibers divide into the right and left bundles. In other cases, the His bundle may run within the muscular part of the membranous septum at the lower portion of the septum. In these cases, identifying and capturing with pacing the His bundle requires a deeper electrode position.

In one aspect, the present disclosure provides a method for His bundle cardiac pacing. In certain embodiments, the method comprises selectively delivering an electrical stimulation pulse to one or more individual electrodes of an approximately linear array of electrodes, wherein the approximately linear array of electrodes is fixated to intersect the His bundle. In certain embodiments, the approximately linear array of electrodes is fixated to cardiac tissue by a tine, an extendible helix, or a combination thereof. In certain embodiments, the approximately linear array of electrodes comprises at least four electrodes. In certain embodiments, the approximately linear array of electrodes is fixated approximately orthogonal to the His bundle.

In certain embodiments, the approximately linear array of electrodes is disposed on a distal section of a pacing lead, such as an AHV lead.

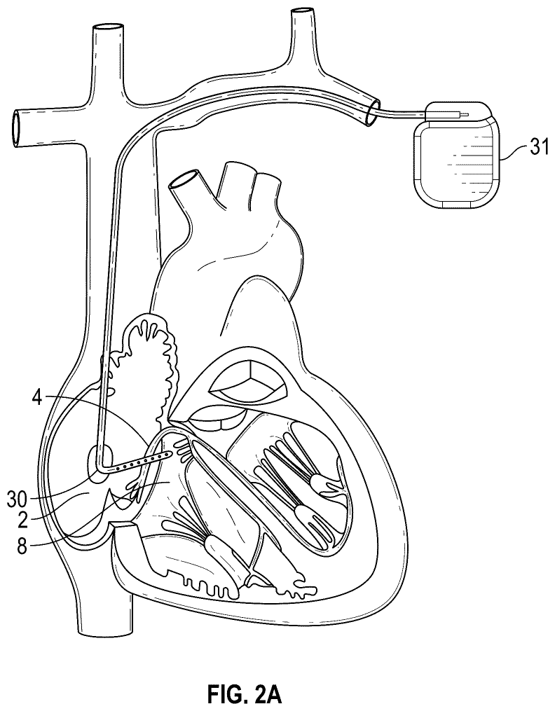

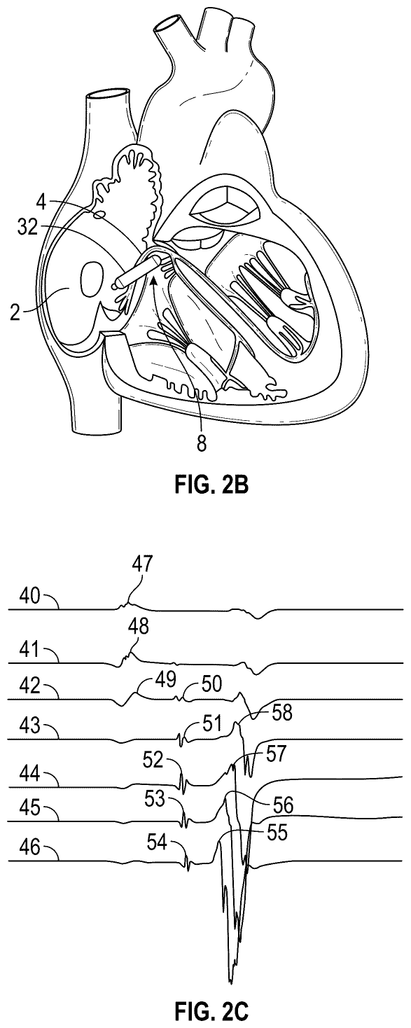

In some such embodiments, the name “AHV lead” was chosen to reflect the regions associated with a properly implanted lead which include the atrium, His bundle, and ventricle. Thus, when the lead is properly implanted, it will allow an attached pacemaker to sense and pace the atrium, His bundle, and ventricles from a single lead. Note that FIG. 2A is a general illustration of an AHV lead 30 across the region of the His bundle connected to a pulse generator 31. The proximal electrodes of the distal section of the AHV lead are adjacent to right atrial tissue 2, the distal electrodes of the distal section of the AHV lead are adjacent to right ventricular tissue 8, and the midsection electrodes of the distal section of the AHV lead are approximately adjacent to the His bundle 4.

In certain embodiments, the approximately linear array of electrodes is disposed on a leadless pacemaker.

In some such embodiments, the regions associated with a properly implanted leadless pacemaker include the atrium, His bundle, and ventricle. Thus, when the linear leadless pacemaker is properly implanted, it will sense and pace the atrium, His bundle, and ventricle. Note that FIG. 2B is a general illustration of a linear leadless pacemaker 32 across the region of the His bundle 4. The proximal electrodes of the linear leadless pacemaker are adjacent to right atrial tissue 2, the distal electrodes of the linear leadless pacemaker are adjacent to right ventricular tissue 8, and the midsection electrodes of the linear leadless pacemaker are approximately adjacent to the His bundle 4.

FIG. 2C illustrates exemplary electrograms which may be acquired from the placement of eight sequential electrodes of the distal section of an AHV lead as depicted in FIG. 2A or a linear leadless pacemaker as depicted in FIG. 2B. The eight electrodes may be combined in sequential pairs to generate the seven-overlapping bipolar electrograms which are shown in 40 to 46. The bipolar electrograms include signals from atrial, His bundle, and ventricular regions of the heart. The proximal electrodes are used to produce the proximal electrograms 40 and 41 which acquire atrial signals 47 and 48, respectively. The mid electrodes are used to produce the mid electrograms 42, 43 and 44. These electrograms include the atrial signal 49, the His bundle potentials 50, 51, and 52, and the ventricular signals 57 and 58. The distal electrodes are used to produce the distal electrograms 45 and 46 which acquire ventricular signals 56 and 55, respectively.

In certain embodiments, the signals depicted in FIG. 2C appear as the physician is in the approximate region of the His bundle prior to deploying the AHV lead or linear leadless pacemaker. The bipolar electrograms in FIG. 2C may be acquired to program the device according to the patient's specific needs. The AHV lead or linear leadless pacemaker may be programmed for sensing or pacing the right atrium, His bundle, and ventricle depending on the acquired bipolar electrogram signals.

### II. Pacing Leads

In another aspect the present disclosure provides a pacing lead. In certain embodiments, the pacing lead comprises a distal section having a plurality of electrodes, said plurality of electrodes arranged in an approximately linear array along a longitudinal axis of the distal section; and a proximal section having an end configured for connection to a pulse generator.

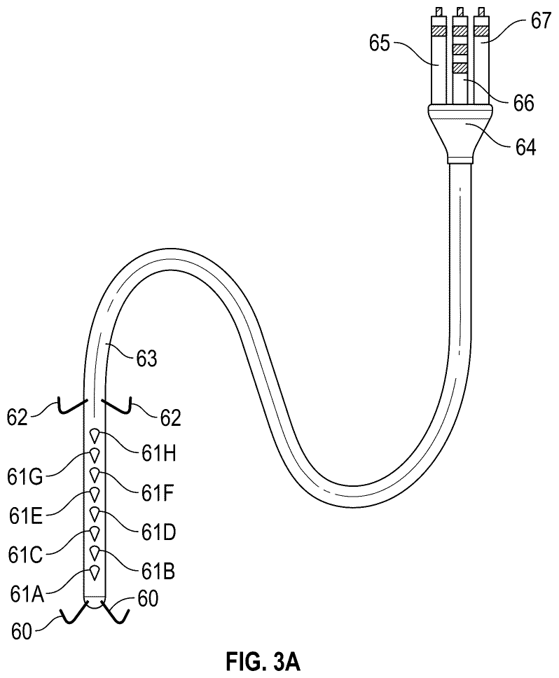

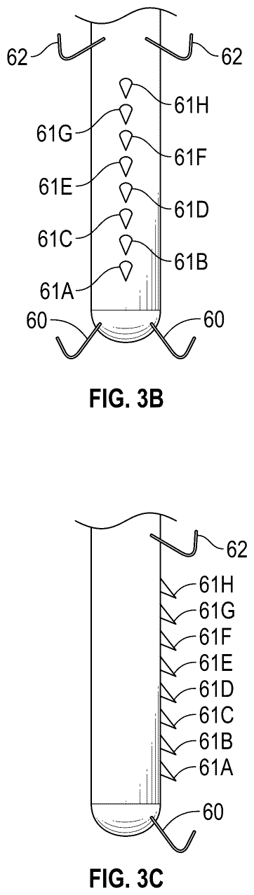

FIG. 3A depicts an exemplary full AHV lead with the distal section of the AHV lead having active tine fixation; the proximal section is adapted to be connected to a pulse generator. Active tine fixation is defined as a fixation mechanism by which the tines penetrate the myocardium upon proper implantation in the heart. The J-shaped flexible tines penetrate the heart upon deployment. The penetrating electrodes function as tines in addition to their sensing and pacing capabilities. The distal section of the AHV lead has eight penetrating electrodes 61A through 61H arranged in an approximately linear array to improve the ability to identify His bundle electrograms. The eight penetrating electrodes serve the purpose of both a fixation mechanism and an electrode. The two pairs of J-shaped flexible fixation tines 60 and 62 serve the purpose of securing the lead and providing an opposing force with respect to the eight penetrating electrodes 61A through 61H. In this context, the word “opposing” refers to the direction of the penetrating electrodes with respect to the pairs of the J-shaped flexible tines. The J-shaped flexible tines are used to grip the myocardium to allow for a secure placement of the distal lead section. The distal section of the AHV lead also has eight penetrating electrodes which serve the purpose of gripping the myocardium in the opposite direction of the J-shaped flexible tines, ultimately providing a secure fixation mechanism. In an alternative embodiment, J-shaped flexible tines may act as both an electrode and a fixation mechanism.

In certain embodiments, each of the eight distal electrodes are electrically connected to independently insulated wires which connects the distal electrodes to proximal electrodes. Proximal electrodes are then connected to a pulse generator. The insulated wires are inside the lead 63. The proximal electrodes are constructed with conventional connector designs which are held together by the yoke 64. The IS-1 connectors are depicted as 65 and 67. The IS-4 connector is depicted as 66. In this design, the two IS-1 and one IS-4 connectors may be attached to a conventional biventricular pacemaker. Thus, to achieve an AHV pacemaker with minimal development of new pulse generators, the AHV lead allows for sensing and pacing of the atrium, His bundle, and ventricle. The conventional connector designs are used to connect to a pulse generator. Those skilled in the art would recognize variations of this electrode design and number of electrodes which generally encompass a distal electrode section, that distal electrode section with electrodes oriented along the side of the lead, and a proximal section that may be adapted to a pulse generator using conventional or unconventional connectors.

FIG. 3B displays a frontal view of the distal section of the exemplary AHV lead having active tine fixation. The two pairs of J-shaped flexible tines 60 and 62 serve the function of a fixation mechanism. The eight penetrating electrodes 61A through 61H act as both a fixation mechanism and an electrode. The two pairs of J-shaped flexible tines synergistically oppose the eight penetrating electrodes, providing a secure fixation mechanism.

The opposing tines can be seen more clearly in FIG. 3C, which displays the side view of the distal section of the exemplary AHV lead having active tine fixation. FIG. 3C shows that the orientation of the eight penetrating electrodes 61A through 61H are in an opposing direction with respect to the two pairs of J-shaped flexible tines 60 and 62. This exemplary design provides a secure fixation mechanism by which the J-shaped flexible tines and penetrating electrodes grip the myocardium. In this exemplary, the eight penetrating electrodes can sense and pace the heart in addition to providing a fixation mechanism.

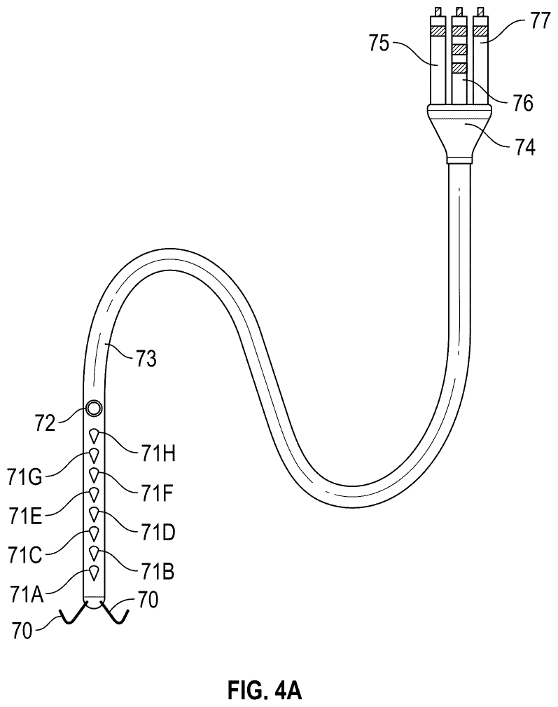

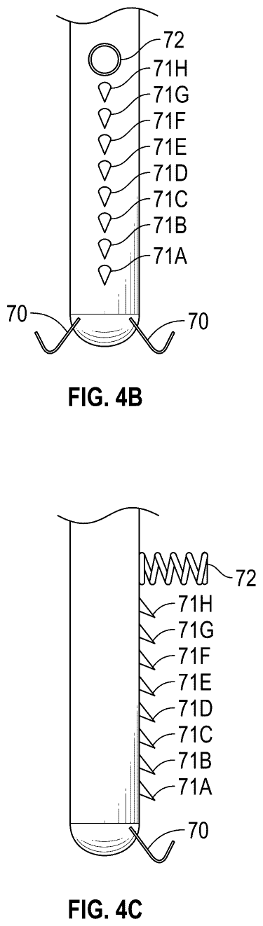

FIG. 4A depicts an exemplary full AHV lead with the distal section of the AHV lead having active tine fixation and a helix fixation mechanism; the proximal section is adapted to be connected to a pulse generator. Active tine fixation is defined as a fixation mechanism by which the tines penetrate the myocardium upon proper implantation in the heart. The helix fixation mechanism is defined as a fixation mechanism by which an extendable helix similar to a cork screw penetrates the myocardium upon proper implantation in the heart. The distal section of this lead has eight penetrating electrodes 71A through 71H arranged in an approximately linear array to improve the ability to identify His bundle electrograms. The eight penetrating electrodes serve the purpose of both a fixation mechanism and an electrode. The pair of J-shaped flexible tines 70 serve the purpose of securing the lead and providing an opposing force with respect to the eight penetrating electrodes 71A through 71H. In this context, the word “opposing” refers to the direction of the penetrating electrodes with respect to the pairs of the J-shaped flexible tines. The pair of J-shaped flexible tines may or may not be staggered depending on the ability of the design to effectively maintain stabilization. This design also contains an active fixation extendable helix 72 in the proximal aspect of the distal section. The helix may be deployed into the right atrium or right atrial septum via clockwise rotation of a pin of the proximal connector. Upon deployment, the helix embeds into the myocardial tissue. The helix acts as a fixation mechanism. In an alternative embodiment, the helix and/or the J-shaped flexible tines may act as both an electrode and a fixation mechanism.

In certain embodiments, each of the eight distal electrodes are electrically connected to independently insulated wires which connects the distal electrodes to proximal electrodes. Proximal electrodes are then connected to a pulse generator. The insulated wires are inside the lead 73. The proximal electrodes are constructed with conventional connector designs which are held together by the yoke 74. The IS-1 connectors are depicted as 75 and 77. The IS-4 connector is depicted as 76. In this design, the two IS-1 and one IS-4 connectors may be attached to a conventional biventricular pacemaker. Thus, to achieve an AHV pacemaker with minimal development of new pulse generators, the AHV lead allows for sensing and pacing of the atrium, His bundle, and ventricle. The conventional connector designs are used to connect to a pulse generator. Those skilled in the art would recognize variations of this electrode design and number of electrodes which generally encompass a distal electrode section, that distal electrode section with electrodes oriented along the side of the lead, and a proximal section that may be adapted to a pulse generator using conventional or unconventional connectors.

FIG. 4B displays a frontal view of the distal section of the exemplary AHV lead having active tine fixation and a helix fixation mechanism. The pair of J-shaped flexible tines 70 penetrate the myocardium and provide secure placement of the lead. The lead is further stabilized by the eight penetrating electrodes 71A through 71H. This design also contains an active fixation extendable helix 72 in the proximal aspect of the distal section which provides further stabilization. The pair of J-shaped flexible tines opposes the eight penetrating electrodes, providing a secure fixation mechanism.

The opposing tines and electrodes can be seen more clearly in FIG. 4C, which displays the side view of the distal section of the exemplary AHV lead having active tine fixation. FIG. 4C shows that the orientation of the eight penetrating electrodes 71A through 71H are in an opposing direction with respect to the pair of J-shaped flexible tines 70. This exemplary design provides a secure fixation mechanism by which the J-shaped flexible tines and penetrating electrodes grip the myocardium. In this exemplary design, the eight penetrating electrodes can sense and pace the heart in addition to providing a fixation mechanism.

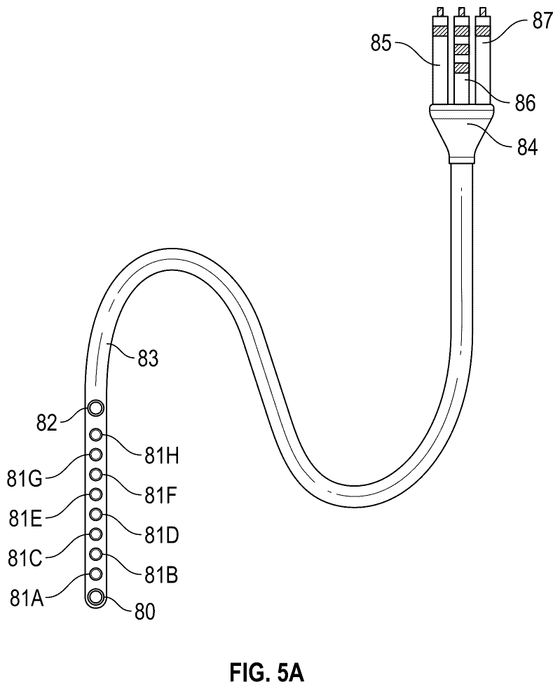

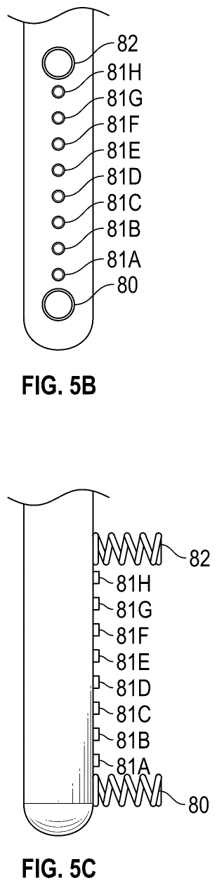

FIG. 5A depicts an exemplary full AHV lead with the distal section of the AHV lead having active helix fixation; the proximal section is adapted to be connected to a pulse generator. The distal section of this lead has two active helix fixation mechanisms 80 and 82 and eight protruding electrodes 81A to 81H. The helix fixation mechanism is defined as a fixation mechanism by which an extendable helix similar to a cork screw penetrates the myocardium upon proper implantation in the heart. The active fixation extendable helix in the proximal aspect of the distal section 82 will be deployed in the right atrial septum via clockwise rotation of a pin of the proximal connector. The active fixation extendable helix in the distal aspect of the distal section 80 will be deployed in the basilar right ventricular septum via clockwise rotation of a pin of the proximal connector. Upon deployment, the helices embed into the myocardial tissue. The eight protruding electrodes 81A to 81H are arranged in an approximately linear array to improve the ability to identify His bundle electrograms. A protruding electrode refers to an electrode that extends or projects from the surface of the lead body. The eight protruding electrodes serve the purpose of sensing and pacing. In an alternative embodiment, the two helices may act as both an electrode and a fixation mechanism; the number of protruding electrodes may be reduced to six such that the combination of the two helices provide an eight-electrode array.

In certain embodiments, each of the eight distal electrodes are electrically connected to independently insulated wires which connects the distal electrodes to proximal electrodes. Proximal electrodes are then connected to a pulse generator. The insulated wires are inside the lead 83. The proximal electrodes are constructed with conventional connector designs which are held together by the yoke 84. The IS-1 connectors are depicted as 85 and 87. The IS-4 connector is depicted as 86. In this design, the two IS-1 and one IS-4 connectors may be attached to a conventional biventricular pacemaker. Thus, to achieve an AHV pacemaker with minimal development of new pulse generators, the AHV lead allows for sensing and pacing of the atrium, His bundle, and ventricle. The conventional connector designs are used to connect to a pulse generator. Those skilled in the art would recognize variations of this electrode design and number of electrodes which generally encompass a distal electrode section, that distal electrode section with electrodes oriented along the side of the lead, and a proximal section that may be adapted to a pulse generator using conventional or unconventional connectors.

FIG. 5B displays a frontal view of the distal section of the exemplary AHV lead having active helix fixation. The distal active helix fixation mechanism 80 and proximal active helix fixation mechanism 82 embed into specific regions of the myocardium through clockwise rotation of a pin of the proximal connector. The six protruding electrodes 81A to 81H mainly serve pacing and sensing functions. The lead configuration can be more readily understood with reference to FIG. 5C which displays the side view of the distal section of the exemplary AHV lead having active helix fixation. The extendable helices 80 and 81 provide a stable fixation mechanism. The six protruding electrodes 81A through 81H mainly serve the purpose of sensing and pacing.

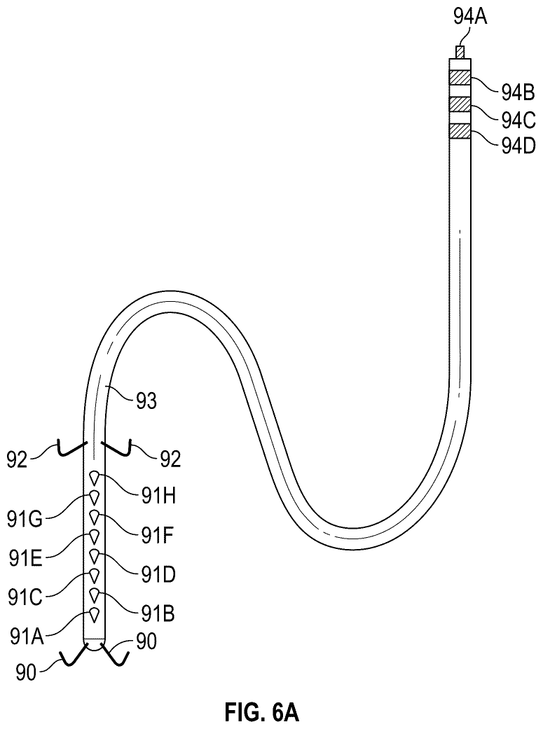

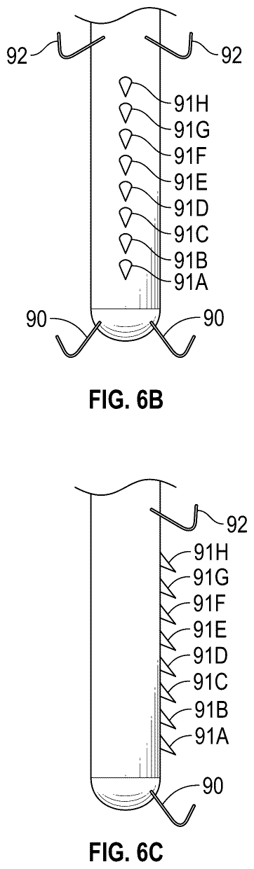

FIG. 6A depicts an exemplary linear pacing lead with the distal section of the linear pacing lead having active tine fixation; the proximal section is adapted to be connected to a pulse generator. Active tine fixation is defined as a fixation mechanism by which the tines penetrate the myocardium upon proper implantation in the heart. The distal section of this lead has four penetrating electrodes labeled 91A, 91B, 91C, and 91D arranged in an approximately linear array to improve the ability to identify His bundle electrograms. The four penetrating electrodes serve a dual purpose of both a fixation mechanism and as electrodes. The pairs of J-shaped flexible tines 90 and 92 serve the purpose of securing the lead and providing an opposing force with respect to the four penetrating electrodes. In this context, the word “opposing” refers to the direction of the penetrating electrodes with respect to the pairs of the J-shaped flexible tines. The J-shaped flexible tines 90 and 92, are used to grip the myocardium to allow for a secure placement of the distal lead section. In alternative embodiments, active fixation tines may also serve as electrodes for sensing and pacing and the number of penetrating electrodes may be modified. Additional embodiments include substituting active tine fixation for an extendable helix.

In certain embodiments, each of the four distal electrodes are electrically connected to independently insulated wires which connects the distal electrodes to proximal electrodes. Proximal electrodes are then connected to a pulse generator. The insulated wires are inside the lead body 93. The proximal electrodes are constructed with a conventional connector design. In this design, one IS-4 connector with electrodes labeled as 94A, 94B, 94C, and 94D may be attached to a conventional biventricular pacemaker. Since the four-electrode design will span over the region of the His bundle, this lead design is focused on His bundle pacing alone rather than pacing the atrium, His bundle, and ventricle. When utilizing the four-electrode design, additional leads may be implanted to allow for sensing and pacing of the right atrium and/or right ventricle. Thus, to achieve an AHV pacemaker with minimal development of new pulse generators, the AHV lead and the linear pacing lead allows for sensing and pacing of the atrium, His bundle, and ventricle or pacing of the His bundle. The conventional connector designs are used to connect to a pulse generator. Those skilled in the art would recognize variations of this electrode design and number of electrodes which generally encompass a distal electrode section, that distal electrode section with electrodes oriented along the side of the lead, and a proximal section that may be adapted to a pulse generator using conventional or unconventional connectors.

FIG. 6B displays a frontal view of the distal section of the exemplary linear pacing lead having active tine fixation. The four penetrating electrodes 91A, 91B, 91C, and 91D oppose the pair of J-shaped flexible tines 90 and 92. The concept of opposing tines can be seen more clearly in FIG. 6C, which displays the side view of the distal section of the linear pacing lead having active tine fixation. This design provides a secure fixation mechanism by which the opposing tines grip the myocardium. In the design presented, the distal electrodes can sense and pace the heart in addition to contributing to the fixation mechanism.

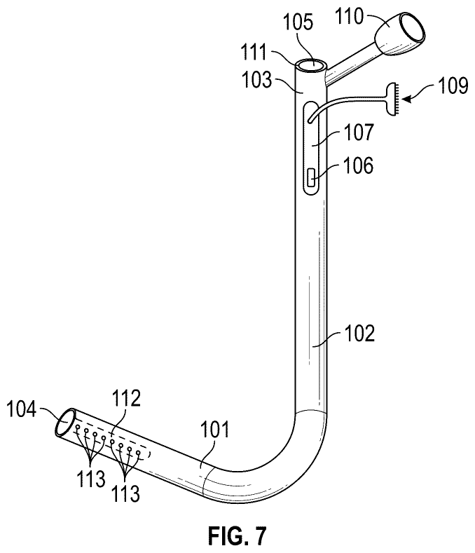

FIG. 7 illustrates an exemplary deployment sheath for implantation of an exemplary linear pacing lead. Control knob 110 is attached to an actuator cable that when twisted results in deflection of the proximal body of the sheath 102 relative to the distal body of the sheath 101. The sheath has a central lumen with a proximal opening 105 and distal opening 104. Electrodes are positioned on a slide mechanism with electrodes labeled 113. When the control lever 106 is pulled proximal, the slider mechanism pulls back the cover with the electrodes 113 to expose the fixation mechanisms of the lead. Once the lead is deployed, the sheath may be pulled back proximal and split at the start of groove 111. The distal electrodes 113 are connected to the proximal connector 109 so that the electrodes may display electrical signals from the heart and facilitate positioning of the sheath for lead deployment.

Alternative forms of the linear pacing lead and the AHV lead may be generated with combinations of any of the following. Note that an approximately linear design was chosen such that the lead may be deployed as a line crossing the conduction system. In essence, the conduction system is also a line and so for proper deployment simply the lines must cross. In traditional His bundle pacing, a point needs to be associated close to the line of conduction which is technically much more difficult. The electrodes need not be oriented exactly in a line and may be staggered or generate another pattern such that the cluster of electrodes may be deployed relative to the elements of the conduction system. Alternative designs of the lead tip may include dome or blunt electrodes that do not penetrate tissue, active tine fixation electrodes in which the tine mechanism penetrates tissue and serves the dual purposes of being both an electrode and a fixation mechanism, or penetrating electrodes which may or may not participate in the fixation mechanism.

The number of electrodes in the linear pacing lead or AHV lead may vary as is practical for being associated with a pacemaker generator. Examples and figures include either four electrodes or eight electrodes. As new connectors are developed, a larger number of electrodes may potentially be deployed such as more than eight electrodes, including ten, twelve, sixteen, or even twenty electrodes. A larger number of electrodes adds complexity to the system but may also allow for identification of improved pacing sites. The lead body may or may not include a lumen. A lumen may be necessary for turning and active fixation mechanism. A passive fixation lead may not require an internal lumen to have fixation deployed. Pacing lead designs with or without lumen are included with this intervention.

### III. Leadless Pacemakers

In another aspect, the present disclosure provides a leadless pacemaker. In certain embodiments, the leadless pacemaker comprises an elongate body having a plurality of electrodes, said plurality of electrodes arranged in an approximately linear array along a longitudinal axis of the elongate body; and a pulse generator

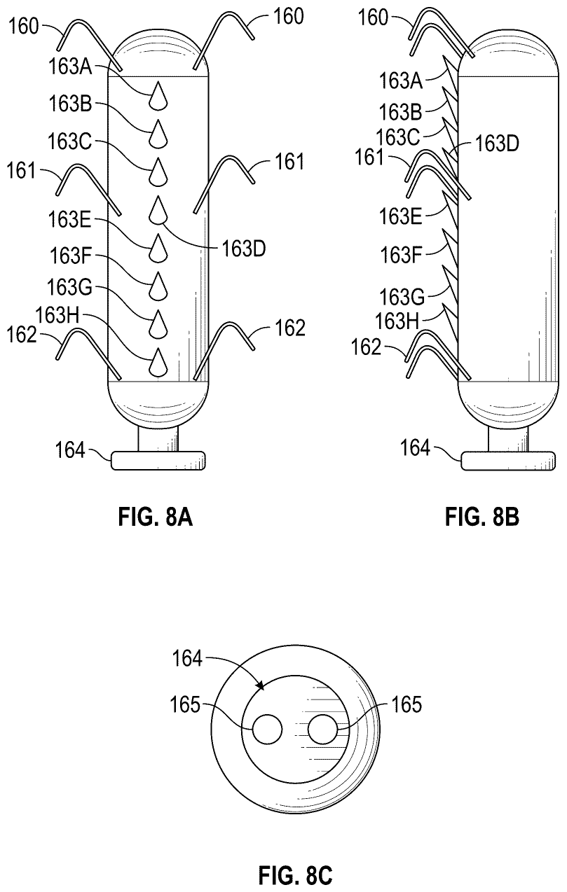

FIG. 8A depicts an exemplary linear leadless pacemaker with the distal section, midsection, and proximal section of the linear leadless pacemaker having active tine fixation. Active tine fixation is defined as a fixation mechanism by which the tines penetrate the myocardium upon proper implantation in the heart. The J-shaped flexible tines penetrate the heart upon deployment. The linear leadless pacemaker has eight penetrating electrodes 163A through 163H arranged in an approximately linear array to improve the ability to identify His bundle electrograms. The eight penetrating electrodes serve the purpose of both a fixation mechanism and an electrode. The three pairs of J-shaped flexible fixation tines 160, 161, and 162 serve the purpose of securing the lead and providing an opposing force with respect to the eight penetrating electrodes 163A through 163H. In this context, the word “opposing” refers to the direction of the penetrating electrodes with respect to the pairs of the J-shaped flexible tines. The J-shaped flexible tines are used to grip the myocardium to allow for a secure placement of the linear leadless pacemaker. The eight penetrating electrodes serve the purpose of gripping the myocardium in the opposite direction of the J-shaped flexible tines, ultimately providing a secure fixation mechanism. In an alternative embodiment, J-shaped flexible tines may act as both an electrode and a fixation mechanism. The entity labeled 164 on the proximal end of the linear leadless pacemaker is the deployment and retrieval knob. Those skilled in the art would recognize variations of this electrode design and number of electrodes which generally encompass a linear leadless pacemaker, electrodes oriented along the side of the pacemaker, and a fixation mechanism that maintains contact of the electrodes to the myocardium.

FIG. 8B displays a side view of the exemplary linear leadless pacemaker having active tine fixation. The concept of opposing tines can be seen more clearly in FIG. 8B, which display the three pairs of J-shaped flexible tines 160, 161, and 162 synergistically opposing the eight penetrating electrodes 161A through 161H. The three pairs of J-shaped flexible tines serve the function of a fixation mechanism while the eight penetrating electrodes act as both a fixation mechanism and an electrode. FIG. 8C illustrates a proximal end view of the exemplary linear leadless pacemaker. The deployment and retrieval knob 164 contain two entities labeled as 165 which are known as the string loop holes. The string loop holes are connected and allow for a loop of string to maintain control of the linear leadless pacemaker during deployment and assessment of position.

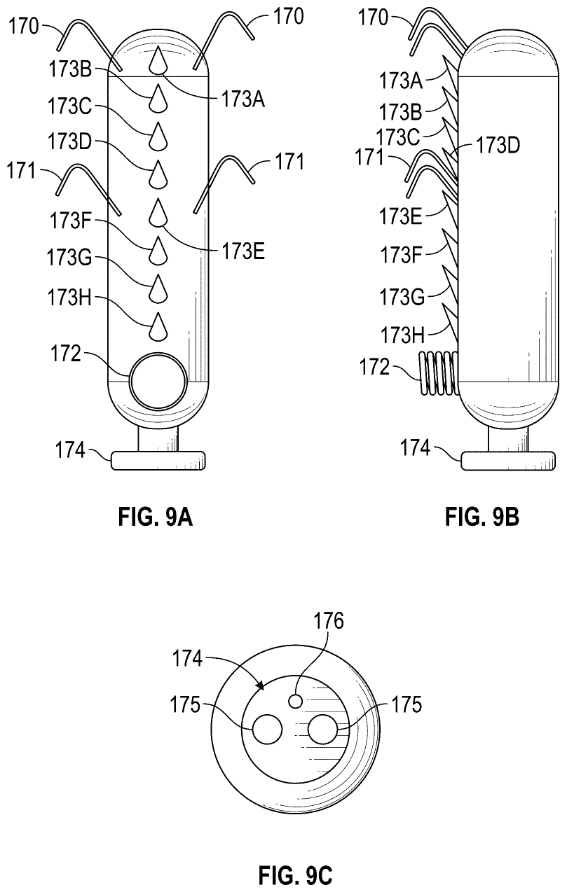

FIG. 9A depicts an exemplary linear leadless pacemaker having active tine fixation and a helix fixation mechanism. Active tine fixation is defined as a fixation mechanism by which the tines penetrate the myocardium upon proper implantation in the heart. The helix fixation mechanism is defined as a fixation mechanism by which an extendable helix similar to a cork screw penetrates the myocardium upon proper implantation in the heart. The linear leadless pacemaker has eight penetrating electrodes 173A through 173H arranged in an approximately linear array to improve the ability to identify His bundle electrograms. The eight penetrating electrodes serve the purpose of both a fixation mechanism and an electrode. The two pairs of J-shaped flexible tines 170 and 171 serve the purpose of securing the lead and providing an opposing force with respect to the eight penetrating electrodes 173A through 173H. In this context, the word “opposing” refers to the direction of the penetrating electrodes with respect to the pairs of the J-shaped flexible tines. The pairs of J-shaped flexible tines may or may not be staggered depending on the ability of the design to effectively maintain stabilization. This design also contains an active fixation extendable helix 172 in the proximal section of the linear leadless pacemaker. The helix may be deployed into the right atrium or right atrial septum via clockwise rotation of a pin of the proximal connector. Upon deployment, the helix embeds into the myocardial tissue. The helix acts as a fixation mechanism. In an alternative embodiment, the helix and/or the J-shaped flexible tines may act as both an electrode and a fixation mechanism. The entity labeled 174 on the proximal end of the linear leadless pacemaker is the deployment and retrieval knob. Those skilled in the art would recognize variations of this electrode design and number of electrodes which generally encompass a linear leadless pacemaker, electrodes oriented along the side of the pacemaker, and a fixation mechanism that maintains contact of the electrodes to the myocardium.

FIG. 9B displays a side view of the exemplary linear leadless pacemaker having active tine fixation and a helix fixation mechanism. The pairs of J-shaped flexible tines 170 and 171 penetrate the myocardium and provide secure placement of the linear leadless pacemaker. The linear leadless pacemaker is further stabilized by the eight penetrating electrodes 173A through 173H. This design also contains an active fixation extendable helix 172 on the proximal aspect of the linear leadless pacemaker which provides further stabilization. The pair of J-shaped flexible tines opposes the eight penetrating electrodes, providing a secure fixation mechanism. FIG. 9C illustrates a proximal end view of the linear leadless pacemaker. The deployment and retrieval knob 174 contain two entities labeled as 175 which are known as the string loop holes. The string loop holes are connected and allow for a loop of string to maintain control of the linear leadless pacemaker during deployment and assessment of position. The deployment and retrieval knob 174 also contain a port 176 through which a stylet will be placed for deployment of the linear leadless pacemaker. In one embodiment, a stylet will be turned to transmit torque to a deployment mechanism such as a gear or motor to turn the extendable helix located inside of the linear leadless pacemaker. In other embodiments, other mechanisms may be used to extend the helix to engage the myocardium.

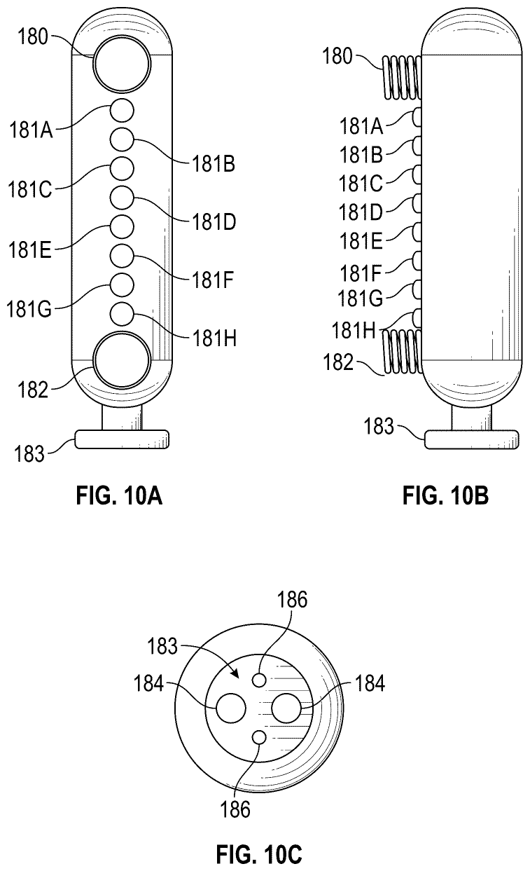

FIG. 10A depicts an exemplary linear leadless pacemaker having active helix fixation. The exemplary linear leadless pacemaker has two active helix fixation mechanisms 180 and 182 and eight protruding electrodes 181A to 181H. The helix fixation mechanism is defined as a fixation mechanism by which an extendable helix similar to a cork screw penetrates the myocardium upon proper implantation in the heart. The active fixation extendable helix in the proximal aspect of the linear leadless pacemaker 182 will be deployed in the right atrial septum via clockwise rotation of a pin of the proximal connector. The active fixation extendable helix in the distal aspect of the linear leadless pacemaker 180 will be deployed in the basilar right ventricular septum via clockwise rotation of a pin of the proximal connector. Upon deployment, the helices embed into the myocardial tissue. The eight protruding electrodes 181A to 181H are arranged in an approximately linear array to improve the ability to identify His bundle electrograms. A protruding electrode refers to an electrode that extends or projects from the surface of the lead body. The eight protruding electrodes serve the purpose of sensing and pacing. In an alternative embodiment, the two helices may act as both an electrode and a fixation mechanism; the number of protruding electrodes may be reduced to six such that the combination of the two helices and six electrodes provide an eight-electrode array. The entity labeled 183 on the proximal end of the linear leadless pacemaker is the deployment and retrieval knob. Those skilled in the art would recognize variations of this electrode design and number of electrodes which generally encompass a linear leadless pacemaker, electrodes oriented along the side of the pacemaker, and a fixation mechanism that maintains contact of the electrodes to the myocardium.

FIG. 10B displays a side view of the distal section of the exemplary linear leadless pacemaker having active helix fixation. The distal active helix fixation mechanism 180 and proximal active helix fixation mechanism 182 embed into specific regions of the myocardium through clockwise rotation of a pin of the proximal connector to provide a fixation mechanism. The six protruding electrodes 181A to 181H mainly serve pacing and sensing functions. FIG. 10C illustrates a proximal end view of the linear leadless pacemaker. The deployment and retrieval knob 183 contain two entities labeled as 184 which are known as the string loop holes. The string loop holes are connected and allow for a loop of string to maintain control of the linear leadless pacemaker during deployment and assessment of position. The deployment and retrieval knob 183 also contain two ports labeled as 186 through which a stylet will be placed for deployment of the linear leadless pacemaker. In one embodiment, a stylet will be turned to transmit torque to a deployment mechanism such as a gear or motor to turn the extendable helices located inside of the linear leadless pacemaker. In other embodiments, other mechanisms may be used to extend the helices to engage the myocardium.

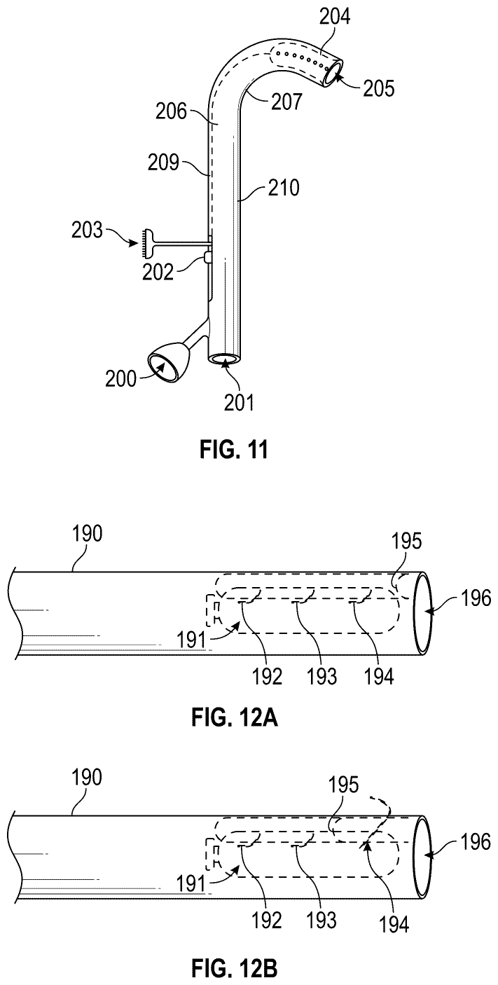

FIG. 11 illustrates a side view of an exemplary delivery sheath for an exemplary linear leadless pacemaker. The distal portion of the delivery sheath is labeled as 207, the distal lumen of the delivery sheath is labeled as 205, and the proximal lumen of the delivery sheath is labeled as 201. The control knob 200 is attached to an actuator cable that when twisted, results in deflection of the distal portion of the delivery sheath 207 relative to the midportion of the delivery sheath 210. The control knob 200 is used to properly position the sheath in the correct location in the His bundle region for proper placement of the linear leadless pacemaker. The distal portion of the delivery sheath 207 has a slider mechanism 204 which contains eight electrodes. The eight electrodes on the slider mechanism serve the purpose of correctly identifying the His bundle region for deployment of the linear leadless pacemaker. The slider mechanism 204 with the eight electrodes are connected via an electrical and mechanical connector 206 to the cable for electrograms 203 and a latch for the sliding mechanism 202. The cable for electrograms 203 may be connected to a recording system or a conventional mapping system and is used to display electrograms to facilitate placement of the linear leadless pacemaker in the region of the His bundle. Once the correct electrograms are displayed to indicate proper placement of the sheath, the linear leadless pacemaker is deployed by withdrawing the slider mechanism 204 via the latch for the sliding mechanism 202 to expose the fixation mechanisms on the linear leadless pacemaker. The outer sheath 209 holds the linear leadless pacemaker in place as the slider mechanism 204 is being withdrawn to expose the linear leadless pacemaker. Upon fixation of the linear leadless pacemaker into the wall of the myocardium, the outer sheath 209 is withdrawn. An inner shaft with a cup at the distal end of the inner shaft is used to hold the linear leadless pacemaker in position as the outer sheath 209 is withdrawn. Once the linear leadless pacemaker has been freed from the delivery sheath system, the linear leadless pacemaker will be assessed for stability. If stability is adequate, then the string that is looped through the string loop holes in the deployment and retrieval knob of the linear leadless pacemaker is cut to release the linear leadless pacemaker from all attachments and complete deployment.

FIG. 12A illustrates a side translucent view of an exemplary linear leadless pacemaker with active tine fixation prior to retraction of the slider 195 during deployment. In FIG. 12A, the entity labeled 191 is the linear leadless pacemaker with active tine fixation which is within the deployment sheath 190. The entity labeled 196 is the distal lumen of the deployment sheath. Prior to retraction of the slider 195, the three pairs of J-shaped flexible tines labeled 192, 193, and 194 are flattened due to the position of the sliding mechanism 195 within the deployment sheath. FIG. 7B illustrates a side translucent view of the linear leadless pacemaker with active tine fixation as the slider 195 is retracted during deployment. As illustrated in FIG. 12B, the pair of J-shaped flexible tines 194 extends out to provide a fixation mechanism in the myocardium upon retraction of the sliding mechanism 195. As the sliding mechanism retracts past the other two J-shaped flexible tines 193 and 192, those J-shaped flexible tines would also be released into the myocardium to provide a fixation mechanism.

Alternative forms of the linear leadless pacemaker may be generated with combinations of any of the following. Note that an approximately linear design was chosen such that the pacemaker may be deployed as a line crossing the conduction system. In essence, the conduction system is also a line and so for proper deployment simply the lines must cross. In traditional His bundle pacing, a point needs to be associated close to the line of conduction which is technically much more difficult. The electrodes need not be oriented exactly in a line and may be staggered or generate another pattern such that the cluster of electrodes may be deployed relative to the elements of the conduction system. Alternative designs of the pacemaker may include dome or blunt electrodes that do not penetrate tissue, active tine fixation electrodes in which the tine mechanism penetrates tissue and serves the dual purposes of being both an electrode and a fixation mechanism, or penetrating electrodes which may or may not participate in the fixation mechanism.

The number of electrodes in the linear leadless pacemaker may vary as is practical for adequate sensing and pacing of regions of the heart. Examples and figures include either four electrodes or eight electrodes. However, a larger number of electrodes may potentially be useful such as more than eight electrodes, including ten, twelve, sixteen, or event twenty electrodes. A larger number of electrodes adds complexity to the system but may also allow for identification of improved pacing sites.

The design describes the external design of the linear leadless pacemaker. Enclosed within are standard pacemaker components including electronics and a battery. In some embodiments, the linear leadless pacemaker is a rechargeable system. In other embodiments, the linear leadless pacemaker may be wired to a power source implanted in the body. External dimensions of the linear leadless pacemaker may be 25 to 45 millimeters in length and 5 to 8 mm in diameter. In other embodiments, the linear leadless pacemaker may be Bluetooth compatible to allow the physician to program and reprogram the device over the course of its lifespan. Other wireless technologies may be used to communicate between the programmer and the linear leadless pacemaker.

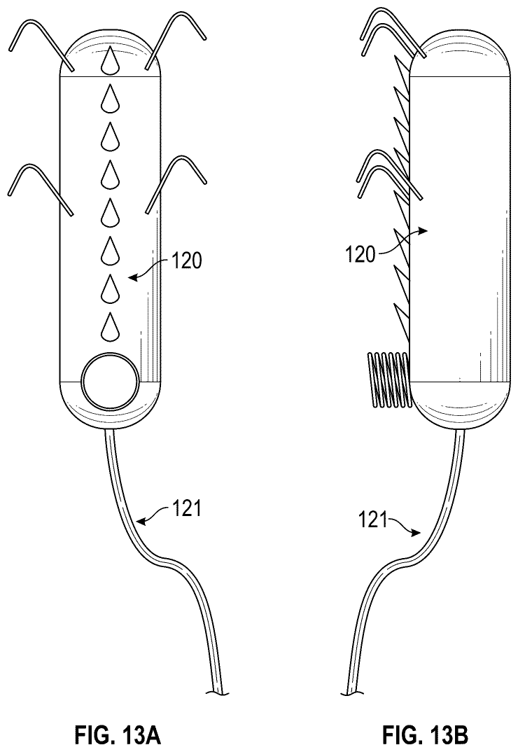

In another aspect of the invention, the linear leadless pacemaker is designed to be connected to a cable that may then be connected to a second device. In this case, the linear leadless pacemaker and cable become a “smart lead.” The smart lead is controlled by the second device for programming and receives energy from the second device. The smart lead may be programmed to utilize a subset of all electrodes for the functions needed for sensing and pacing. The advantage of the smart lead is that the connecting cable may be simplified and require a fewer number of insulated wires than the number of electrodes on the smart lead. FIG. 13A is a frontal view and FIG. 13B is a side view of the distal aspect of the smart lead. The body of the smart lead 120 is similar to the linear leadless pacemaker design in terms of electrodes and fixation mechanisms. The cable for the smart lead is labeled 121 in FIG. 13A and FIG. 13B.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

While certain of the preferred embodiments of the present invention have been described and specifically exemplified above, it is not intended that the invention be limited so such embodiments. Various modifications may be made thereto without departing from the scope of the present invention.

What is claimed is:

1. A method for His bundle cardiac pacing, the method comprising:
selectively delivering an electrical stimulation pulse to one or more individual electrodes of an approximately linear array of electrodes, wherein the approximately linear array of electrodes is fixated to intersect the His bundle.

2. The method of claim 1, wherein the approximately linear array of electrodes is fixated to cardiac tissue by a tine, an extendible helix, or a combination thereof.

3. The method of claim 1, wherein the approximately linear array of electrodes comprises at least four electrodes.

4. The method of claim 1, wherein the approximately linear array of electrodes is fixated approximately orthogonal to the His bundle.

5. The method of claim 1, wherein the approximately linear array of electrodes is disposed on a distal section of a pacing lead.

6. The method of claim 1, wherein the approximately linear array of electrodes is disposed on a leadless pacemaker.

7. A pacing lead comprising: a distal section having a plurality of electrodes, said plurality of electrodes arranged in an approximately linear array along a longitudinal axis of the distal section; and a proximal section having an end configured for connection to a pulse generator.

8. The pacing lead of claim 7, wherein the distal section further comprises one or more fixation elements.

9. The pacing lead of claim 8, wherein the one or more fixation elements includes at least one tine, at least one helix, or a combination thereof.

10. The pacing lead of claim 7, wherein the approximately linear array of electrodes comprises at least four electrodes.

11. The pacing lead of claim 7, wherein the approximately linear array of electrodes comprises at least eight electrodes.

12. The pacing lead of claim 7, wherein the approximately linear array of electrodes comprises at least one penetrating electrode and/or at least one active tine electrode.

13. The pacing lead of claim 7, wherein at least one electrode of the plurality of electrodes is configured to act as a fixation element.

14. A leadless pacemaker comprising: an elongate body having a plurality of electrodes, said plurality of electrodes arranged in an approximately linear array along a longitudinal axis of the elongate body; and a pulse generator.

15. The leadless pacemaker of claim 14, further comprising one or more fixation elements.

16. The leadless pacemaker of claim 15, wherein the one or more fixation elements includes at least one tine, at least one helix, or a combination thereof.

17. The leadless pacemaker of claim 14, wherein the approximately linear array of electrodes comprises at least four electrodes.

18. The leadless pacemaker of claim 14, wherein the approximately linear array of electrodes comprises at least eight electrodes.

19. The leadless pacemaker of claim 14, wherein the approximately linear array of electrodes comprises at least one penetrating electrode and/or at least one active tine electrode.

20. The leadless pacemaker of claim 14, wherein at least one electrode of the plurality of electrodes is configured to act as a fixation element.
